(19) United States
(12) Patent Application Publication
(10) Pub. No.: US 2022/0361861 A1
(43) Pub. Date: Nov. 17, 2022

(54) CONCENTRIC CORE PUNCTURE LOCATING SYSTEM

(71) Applicant: Teleflex Life Sciences Limited, Valletta (MT)

(72) Inventors: Joseph Todd Grintz, Glenmoore, PA (US); Gabrielle Herrmann, Glenside, PA (US)

(73) Assignee: Teleflex Life Sciences Limited, Valletta (MT)

(21) Appl. No.: 17/662,496

(22) Filed: May 9, 2022

Related U.S. Application Data
(60) Provisional application No. 63/187,627, filed on May 12, 2021.

(51) Int. Cl. A61B 17/00

(52) U.S. Cl. CPC A61B 17/0057; A61B 2017/00654; A61B 2017/00637; A61B 2017/00672; A61B 2017/00623

(57) ABSTRACT

Disclosed are a puncture sealing device and methods of locating a puncture site within a vessel. The systems can include elongated dilators and access sheaths that are configured to locate the puncture site within a vessel so that the position of the puncture site relative to a distal end of the access sheath is known during a puncture sealing procedure.

### CROSS-REFERENCE TO RELATED APPLICATIONS

This application claims the benefit of and priority to, under 35 U.S.C. § 119(e) of U.S. Provisional Application No. 63/187,627 filed on May 12, 2021, the entire contents of which is incorporated herein by reference.

### TECHNICAL FIELD

The present application relates to a puncture locating system and method, and in particular, to a concentric core puncture location system utilized in a vascular closure device.

### BACKGROUND

During the use of vascular closure systems after vascular interventions, it is often important to know the location of a puncture in the vessel, and in particular, providing for exact placement of vascular sheaths. Typically, a “blood flashback” method is used to position a vascular device, but this technique is not feasible with catheters whose size is similar to the vessel internal diameter (ID) due to the limited flow possible.

Puncture locating dilators have been utilized to determine the location of the puncture in the vessel, however larger puncture locating dilators are desired. Increasing the size on current dilators can result in the dilators having decreased flexibility and increased stiffness. Utilizing different materials to provide increased flexibility, however, can make it difficult to provide visual markings on the body of the dilator to measure the depth of the arteriotomy

### SUMMARY

There is a need to provide a larger puncture locating dilator that is flexible and also enables the dilator to be visibly marked in order to measure the location of the puncture in a vessel. An embodiment of the present disclosure includes a puncture location device. The puncture location device is configured to determine a location of a puncture in an artery relative to a skin surface of a patient. The puncture location device includes a flexible elongated body that extends along a central longitudinal axis. The flexible elongated body includes a proximal end, a distal end spaced from the proximal end along the central longitudinal axis, an outer layer, an inner channel that extends from the proximal end toward the distal end along the central longitudinal axis, at least one distal port that is open to the inner channel and that extends through the outer layer and the inner core, and a proximal port open to the inner channel and positioned between the proximal end and the at least one distal port. The flexible elongated body further includes an inner core that defines the inner channel. The inner core is surrounded by the outer layer and includes a first polymeric material. The outer layer includes a second polymeric material that is different from the first polymeric material. The at least one distal port is configured to receive blood therethrough such that blood travels through the inner channel and through the proximal port when the at least one distal port is placed in a path of blood flow.

### BRIEF DESCRIPTION OF THE DRAWINGS

The foregoing summary, as well as the following detailed description, will be better understood when read in conjunction with the appended drawings. The drawings show illustrative embodiments of the disclosure, in which there is shown in the drawings example embodiments for the purposes of illustration. It should be understood, however, that the application is not limited to the precise arrangements and instrumentalities shown.

FIG. 1 is a perspective view of a puncture locating dilator according to an embodiment of the present disclosure;

FIG. 2 is a cross-section of the puncture locating dilator taken along line B-B in FIG. 1;

FIG. 3 is a cross-section of the puncture locating dilator taken along line A-A in FIG. 1;

FIG. 4 is a side view of the puncture locating dilator shown in FIGS. 1-3;

FIG. 5 is a top plan view of the puncture locating dilator shown in FIGS. 1-4;

FIG. 6 is an enlarged perspective view of the proximal end of the puncture locating dilator shown in FIGS. 1-5;

FIG. 7 is a perspective view of the puncture locating dilator shown in FIGS. 1-6 with a vascular closure device in accordance with an embodiment of the present disclosure;

FIG. 8A is a perspective view of the vascular closure device shown in FIG. 7;

FIG. 8B is a perspective view of the vascular closure device shown in FIG. 8A with a portion of the housing removed;

FIG. 8C is a is a perspective view of a sealing device associated with the vascular closure device shown in FIGS. 8A and 8B;

FIG. 8D is a side sectional view of a distal portion of the vascular closure device shown in FIGS. 8A-8C;

FIG. 9A is a schematic showing the puncture locating dilator shown in FIGS. 1-7 positioned such that the distal port is disposed within a vessel proximate to a vessel puncture and at least one of the markings being visible above the surface of the skin;

FIG. 9B is a schematic showing the access sheath shown in FIG. 7 being moved into the vessel;

FIG. 9C is a schematic showing a sheath dilator removed from the access channel and the sheath body positioned such that a depth marking on the sheath body that corresponds to the at least one marking on the puncture locating dilator is visible above the surface of the skin;

FIG. 10A is a schematic showing the access sheath shown in FIG. 7, positioned such that the distal port is disposed within a vessel proximate to a vessel puncture and at least one of the markings being visible above the surface of the skin;

FIG. 10B is a schematic showing the access sheath of FIG. 10A, moved further into the vessel such that a full insertion marker on the sheath body that is proximal to the plurality of markings is adjacent the patient's skin;

FIG. 10C is a schematic showing the access sheath of FIG. 10B with the sheath dilator removed from the access channel and a closure device moved into the access channel; and

FIG. 10D is a schematic showing the access sheath of FIG. 10C moved proximally such that the at least one marking noted in FIG. 8A is again visible above the surface of the skin.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

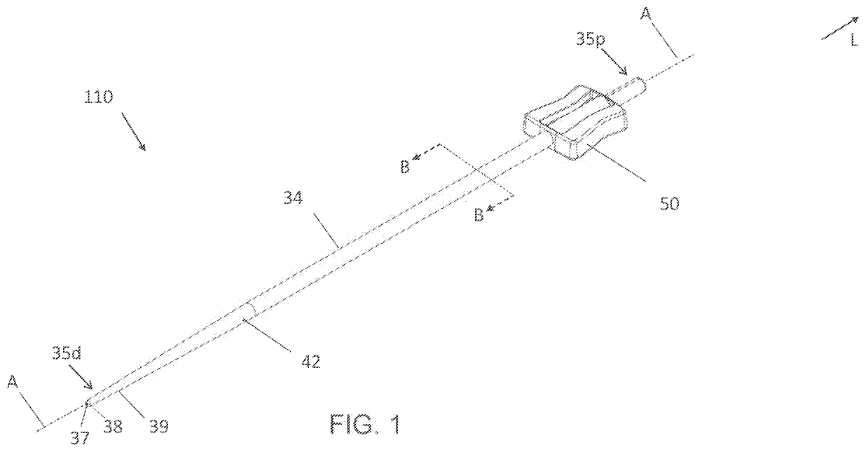

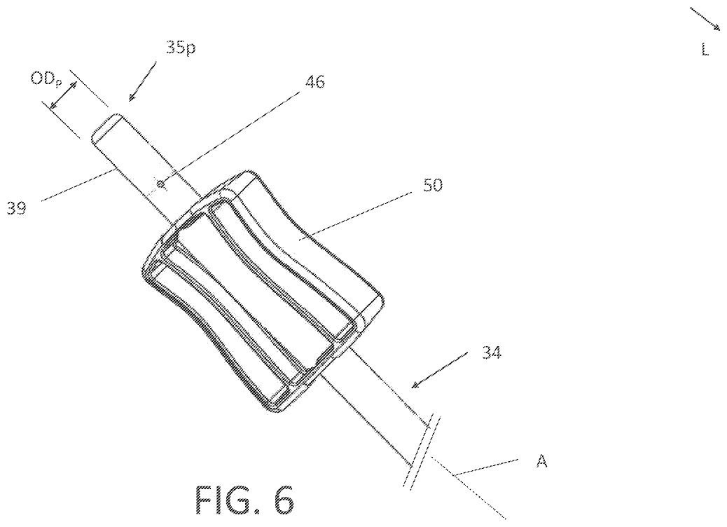

Referring to FIGS. 1-7, a puncture sealing system in accordance with an embodiment of the invention can include a puncture locating dilator 110 that is configured to locate a puncture site in a vessel of a patient. The puncture locating dilator 110 may be used in locating a puncture in a vessel during or along with an interventional cardiovascular procedure. In the illustrated embodiment, the puncture locating dilator 110 has a dilator size of 14F.

Typically, before an interventional cardiovascular procedure, a puncture may be made in the femoral artery. In one example, a vascular closure device composed of an absorbable anchor, a folding sealing plug, a suture and a downward locking member have been developed and may be used to seal these punctures. However, before sealing can occur the depth at which the device needs to be inserted must be attained. Currently, the procedure is conducted with a puncture locator comprising of at least one distal port towards the distal end and one outlet opening at the proximal end. Conventional puncture locators allow blood to flow through the dilator and out an outlet opening when the distal port is present within the circulatory tract.

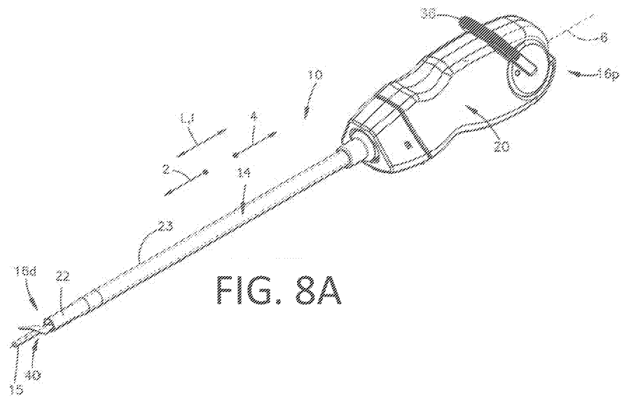

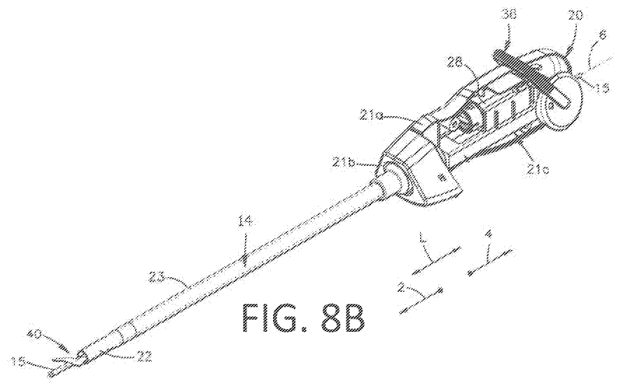

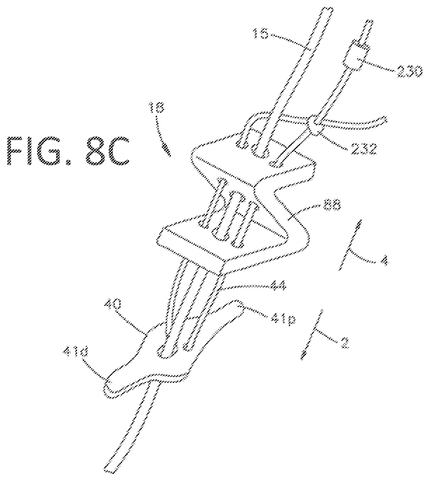

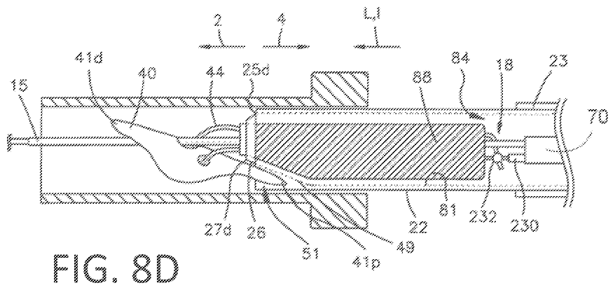

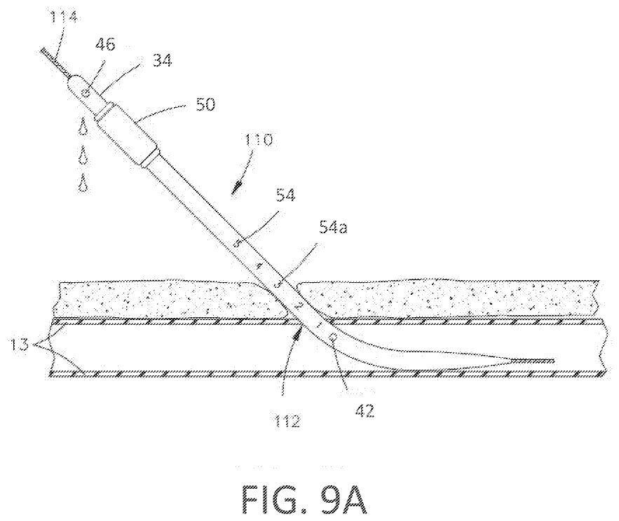

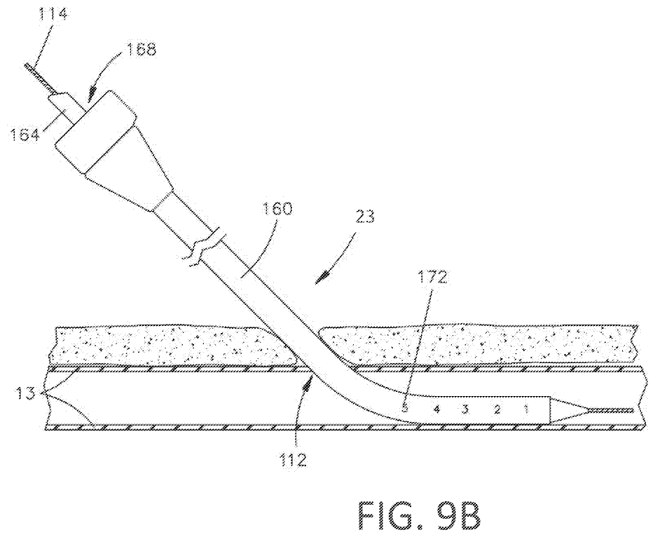

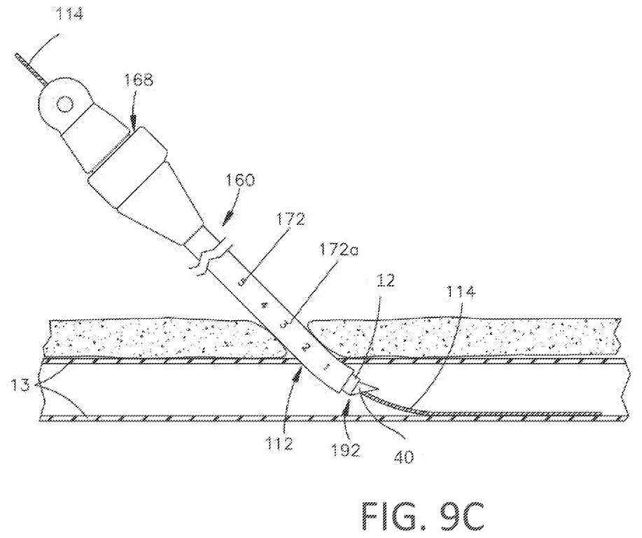

Continuing with FIGS. 1-7, the puncture locating dilator 110 is an elongated dilator configured to locate a puncture site 112 (FIGS. 8A-9D) in a vessel 13 (FIGS. 8A-9D). The puncture locating dilator 110 includes a flexible elongated body, or dilator body 34 that is elongated along a central longitudinal axis A in a first direction L. The first direction L is parallel to the central longitudinal axis A and may be referred to as a longitudinal direction in this disclosure. The dilator body 34 defines a proximal end 35p and a distal end 35d that is spaced from the proximal end 35p along the first direction L. The distal end 35d of the dilator may be tapered to facilitate entry into the vessel.

The dilator body 34 further defines an inner core 37, which may define an inner channel, or guide channel 38 that extends from the proximal end 35p toward the distal end 35d along the central longitudinal axis A, and an outer layer 39 that surrounds the inner core 37. The outer layer 39 defines an outer cross-sectional dimension that is substantially perpendicular to the central longitudinal axis A. The inner core 37 is made of a first polymeric material. In the illustrated embodiment, the first polymeric material contains a predominant amount of low-density polyethylene. In alternative embodiments, the first polymeric material may contain varying amounts of low-density polyethylene. The outer layer 39 is made of a second polymeric material that is different from the first polymeric material. In the illustrated embodiment, the second polymeric material contains a predominant amount of high-density polyethylene. In alternative embodiments, the second polymeric material may contain varying amounts of high-density polyethylene. The inner core 37 has a greater flexibility than the outer layer 39. This configuration allows the inner core 37 to be flexible, while the thin outer layer 39 is stiffer, allowing for the outer layer 39 to be laser-marked. Thus, the desired flexibility and stiffness of the dilator 110 is maintained.

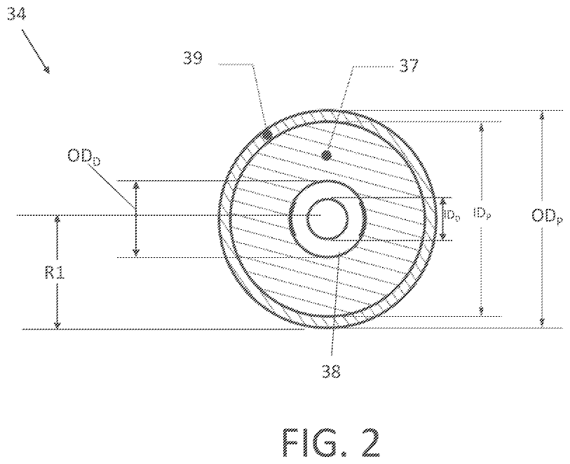

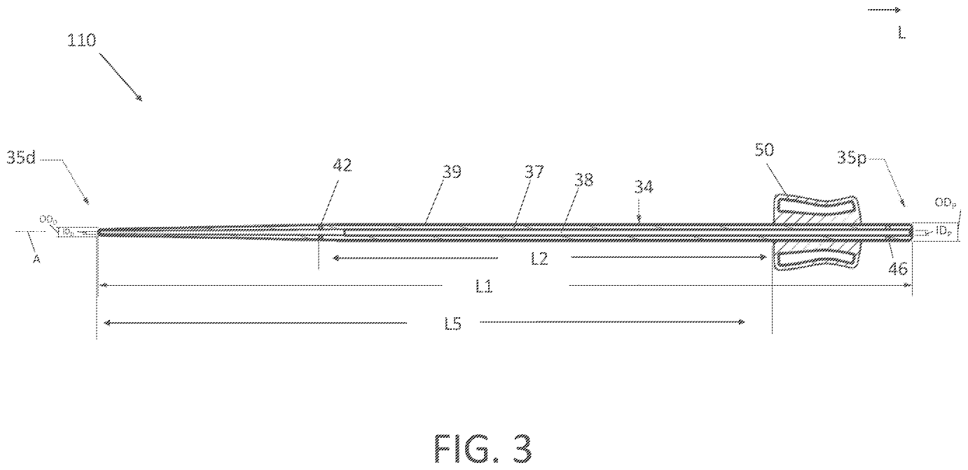

Referring to FIGS. 2 and 3, the puncture locating dilator 110 is configured to be moved along a guide wire 114 (not depicted) toward the puncture site 112 (not depicted) such that the puncture locating dilator 110 enters the vessel 13 (not depicted) through the puncture site 112 (not depicted). As the puncture locating dilator 110 enters the vessel 13 the puncture locating dilator 110 dilates the puncture site 112. The inner channel 38 extends through the dilator body 34 along the first direction L from the distal end 35d through to the proximal end 35p. The inner channel 38 is configured to receive the guide wire 114 such that the puncture locating dilator 110 can be moved along the guide wire 114 toward the puncture site 112.

The puncture locating dilator 110 is sized for a range of procedures. The elongated body 34 has a length L1. In the illustrated embodiment, the length L1 is approximately 9.375 inches. In another embodiment, the length L1 is at least 9.365 inches. The flexible elongated body 34 of the puncture locating dilator 110 includes an outer cross-sectional diameter OD of the outer layer 39 and an inner cross-sectional diameter ID of the inner core 37.

As described above, the distal end 35d of the dilator may be tapered to facilitate entry into the vessel. Thus, the proximal end 35p of the inner core 37 has a proximal inner diameter ID_{P} and a proximal outer diameter OD_{P}, while the distal end 35d of the inner core 37 has a distal inner diameter ID_{D} and a distal outer diameter OD_{D}. In the illustrated embodiment, the proximal inner diameter ID_{P} may range from approximately 0.035 inches to approximately 0.038 inches. The proximal outer diameter OD_{P} is at least approximately 0.18 inches. Additionally, in the illustrated embodiment, the distal inner diameter ID_{D} may range from approximately 0.035 inches to approximately 0.037 inches.

The flexible elongated body 34 includes a radius R1 measured from the outer layer 39 to the center of the inner core 37. In one example, the outer layer 39 comprises between approximately 0.085 inches and approximately 0.0115 inches of the radius R1 of the dilator body 34. In another embodiment, the outer layer 39 comprises about 0.01 inches of the radius R1 of the flexible elongated body 34. In another embodiment, the inner core 37 comprises between approximately 0.0440 and approximately 0.060 inches of the radius R1 of the flexible elongated body 34. In another example, the inner core 37 is about 0.052 of the radius of the flexible elongated body 34.

Further, in one example, the inner core 37 and the outer layer 39 comprise between 65% and 80% of the outer diameter OD of the flexible elongated body. In another example, the inner core 37 and the outer layer 39 comprise about 75% of the outer diameter OD of the flexible elongated body 34. In a further example, the outer layer 39 comprises about up to about 10% of the outer diameter OD of the flexible elongated body 34. The outer layer 39 may comprise between about 3% and 8% of the outer diameter OD of the flexible elongated body 34. The inner core 37 may also comprise between about 20% and 35% of the outer diameter OD of the flexible elongated body 34. The inner core may comprise between about 25% and 30% of the outer diameter OD of the flexible elongated body 34. However, dimensions outside of these ranges are possible.

Referring to FIGS. 3-6, the dilator 110 can further define at least one distal port 42 and a proximal port 46. The distal port 42 is open to the inner channel 38 and extends through the outer layer 39 and the inner core 37. The proximal port 46 is open to the inner channel 38 and positioned between the proximal end 35p and the at least one distal port 42. In the illustrated embodiment, the diameter of the distal port 42 is 0.030 inches and the diameter of the proximal port 46 is approximately 0.032 inches. In alternative embodiments, the diameter of the distal port 42 is at least 0.025 inches and the diameter of the proximal port is at least 0.029 inches.

The distal port 42 and the proximal port 46 are in fluid communication with each other such that when the distal port 42 enters the vessel 13, blood from the vessel 13 will enter the distal port 42, travel through the inner channel 38, and exit the proximal port 46, to thereby indicate that the distal port 42 has entered the vessel 13, as further explained below. In this way, a position of the puncture site 112 can be located or otherwise determined. In the illustrated embodiment, distal port 42 and the proximal port 46 extend into the inner channel 38 such that blood entering the distal port 42 will travel through the inner channel 38, around the guide wire 114, and out the proximal port 46. It should be appreciated, however, that in some embodiments, the inner channel 38 and the channel through which the blood flows can be separate and distinct from each other, as desired.

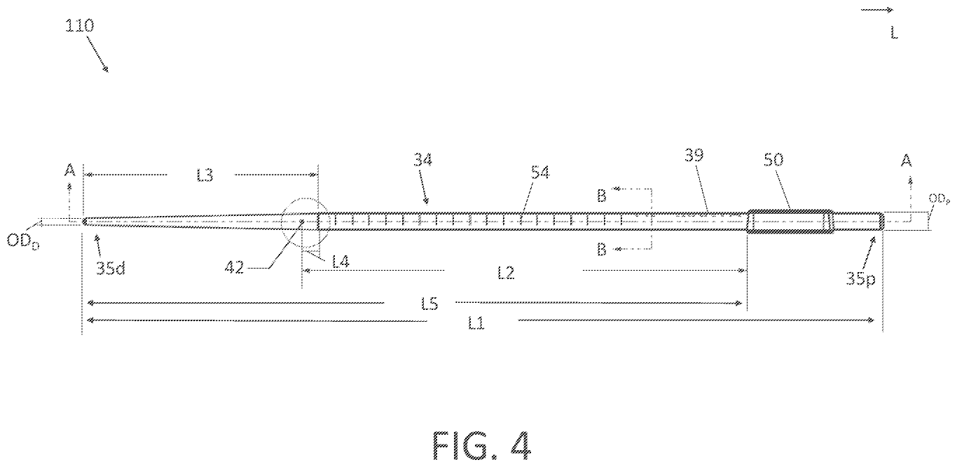

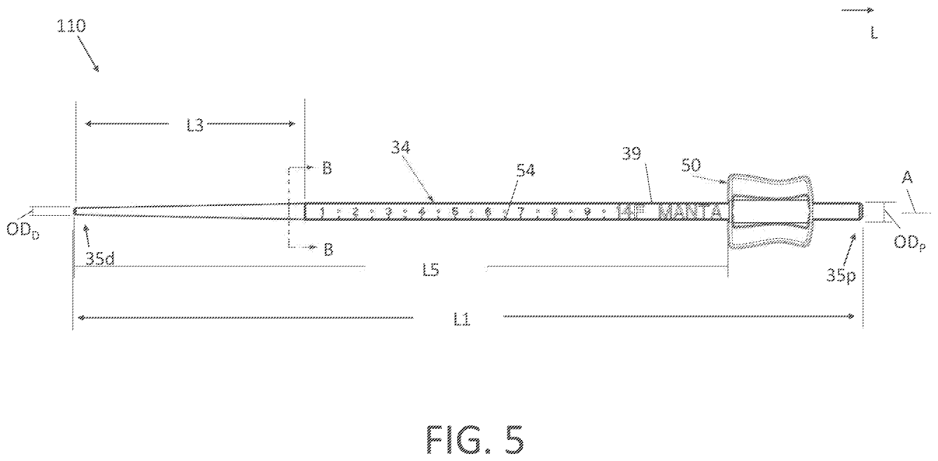

Referring to FIGS. 4 and 5, the puncture locating dilator 110 can further include a plurality of depth markings 54 spaced from each other along the first direction L between the distal port 42 and proximal port 46. The depth markings 54 can be used to visually note the depth or otherwise the location of the puncture site 112 of the vessel 13 when the puncture locating dilator 110 has been positioned within the vessel. In the illustrated embodiment, the plurality of depth markings 54 are etched into the outer surface of the outer layer 39 and are not etched into the inner core 37. In the illustrated embodiment, the plurality of markings are etched onto the outer layer 39 such that the length L4 from the center of the distal port 42 to a first marking 54a of the plurality of depth markings 54 is approximately 0.394 inches. The length L3 from the distal end 35d to the first marking 54a is approximately 2.75 inches. In alternative embodiments, the length L4 from the center of the distal port to the first marking may range from approximately 0.391 inches to approximately 0.397 inches. In addition, in the illustrated embodiment, the length between each of the plurality of depth markings 54 is approximately 0.197 inches. In alternative embodiments, the length between the depth markings 54 may range from approximately 0.194 inches to approximately 0.200 inches. In the illustrated embodiment, the depth markings 54 are numbers aligned on the dilator body 34 along the central axis A. It should be appreciated, however, that the depth markings 54 can have other configurations as desired. For example, the depth markings can be configured as symbols as desired.

The depth markings 54 can be used to locate the puncture site 112. That is, after a position of the puncture site 112 has been located with the distal port 42, a position of a first visible marking of the plurality of depth markings 54 on the dilator 110 that is adjacent the patient's skin can be noted when the blood flows. Therefore, the position of the puncture site 112 can be known for the remainder of the procedure. The noted first depth marking 54a can be noted with a sticker that is placed directly on the patient's skin as desired. It should be appreciated, however, that the first depth marking 54a can be noted using other configurations as desired. For example, the first depth marking 54a can be noted with a tag, card, clip, etc. In an alternative embodiment, the depth markings 54 of this embodiment can either be used alone or in combination with radiopaque markers.

Referring to FIGS. 1-6, the puncture locating dilator 110 can further include a hub 50 that extends radially out from the dilator body 34 between the inlet and proximal ports 42 and 46. The hub 50 can be configured as a handle that can be firmly grasped to thereby move the puncture locating dilator 110 along the guide wire 114. It should be appreciated, however, that the hub 50 can be located anywhere along the dilator body 34 as desired. In the illustrated embodiment, the hub 50 has a length of approximately 1.02 inches, a width of approximately 0.89 inches, and a thickness of approximately 0.26 inches. In alternative embodiments, the dimensions of the hub 50 may vary. In the illustrated embodiment, the length L2 between the distal port 42 and the hub 50 is approximately 5.213 inches. The length L5 between the distal end 35d and the hub 50 is approximately 7.771 inches. In an alternative embodiment, the length L2 between the distal port 42 and the hub 50 is at least 5.212 inches. The length L5 between the distal end 35d and the hub 50 is between approximately 7.756 inches and approximately 7.786 inches.

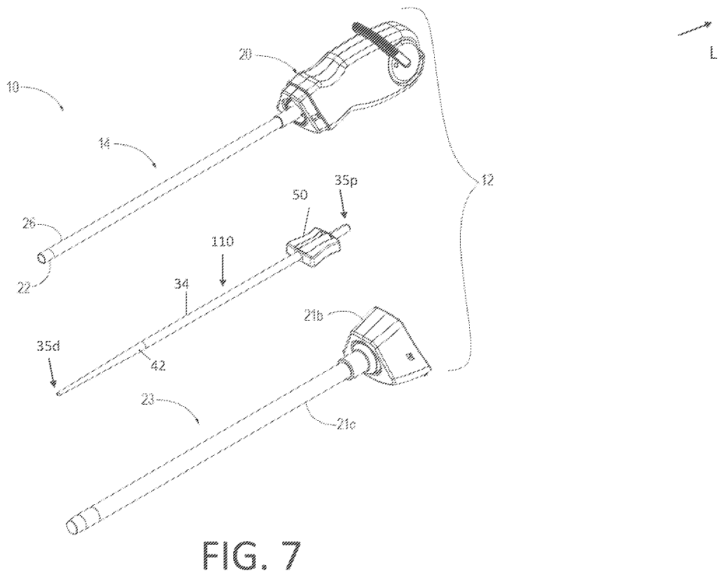

Referring to FIG. 7, in the illustrated embodiment, the puncture locating dilator 110 described herein is used in connection with a vascular closure system 10 to determine puncture location during vascular closure procedures. In alternative embodiments, the puncture locating dilator 110 described herein may be used generally for puncture location in a vessel 13. As shown in FIG. 7, the puncture sealing system can further include an access sheath 23 that is also configured to be moved along the guide wire 114 toward the puncture site 112 and into the vessel 13 so as to further dilate the puncture site 112 and subsequently provide access to the vessel 13. The access sheath 23 can then receive a sealing device that is configured to seal the puncture site 112. It should be appreciated, however, that the system can include additional dilators that have cross-sectional diameters that are different (e.g. greater) than the diameter of the puncture locating dilator 110 but less than that of the access sheath 23 so that the puncture site 112 can be gradually dilated and prepared for the access sheath 23.

Continuing with FIGS. 7-8D, the vascular closure system 10 includes a closure device 12 that is configured to seal a puncture in a vessel wall. The puncture locating dilator 110 is configured to facilitate placement of the closure device 12 into the desired position within a puncture site of a vessel wall following a surgical procedure. The closure device 12 includes a deployment assembly 14 and an access sheath 23. The access sheath 23 can be inserted into the vessel and the deployment assembly 14 can be inserted into the access sheath 23 to position a sealing unit 18 (FIG. 8C) into the vessel.

Referring to FIGS. 8A and 8B, a vascular closure device 12 includes a sealing unit 18 at least partially disposed within a deployment assembly 14. The vascular closure device 12 can be configured such that after a distal portion of deployment assembly 14 is inserted through a puncture site of the vessel, the sealing unit 18 is deployed to thereby seal or otherwise close the puncture site of the vessel. The deployment assembly 14 is configured to control orientation of a toggle 40 of the sealing unit 18 in an easier and more efficient manner during deployment of the sealing unit 18. Furthermore, the deployment assembly 14 is configured to reduce forces required to deploy the sealing unit 18 and seal the puncture.

In accordance with the illustrated embodiment, the deployment assembly 14 includes a release component 22 that restrains the toggle 40, a delivery component 26 (See FIG. 8D) that contains at least a portion of the toggle 40 and a suture 43 of the sealing unit 18, a guide member 15, and one or more actuators 36 coupled to the release component 22. The deployment assembly 14 may also include a tamper 70, in the form a tube, that extends along the suture 43 and is located in a proximal direction relative to the locking member 230 (See FIG. 8D). The guide member 15 extends through the sealing unit 18 and is configured to receive a guide wire as will be discussed below. In another example, the deployment assembly 14 can be configured so that the guide wire 114 extends along the side of the toggle 40. The release component 22 is operatively associated with the suture 43 such that actuation of the actuator 36 causes the release component 22 to 1) release the toggle 40, and 2) apply tension to the suture 43, which urges the toggle 40 against the delivery component 26 and orients the toggle 40 in the sealing position. The guide member 15 is configured to be removed from at least the sealing unit 18 prior to the sealing unit 18 sealing the puncture.

Turning to FIG. 8C, the sealing unit 18 includes the toggle 40 connected to the suture 43, a plug 88 coupled to the suture 43 and spaced from the toggle 40 in a proximal direction 4, and a locking member 230 proximal to the plug 88. The toggle 40 includes a distal end 45 d and a proximal end 41 p opposite to the proximal end 41 p, and a plurality of apertures (not numbered) extending therethrough. The suture 43 extends through the apertures as illustrated such that an end of the suture 43 is formed into a slidable knot 232. The knot 232 is slidable along the suture 43 between the plug 88 and the locking member 230. In an implanted state, the toggle 40 is adjacent to an inner surface of the vessel and the locking member 230 squeezes the toggle 40 and the plug 88 against the vessel to seal the puncture.

The sealing unit 18 is formed with materials suitable for surgical procedures such as any biocompatible material. It should be appreciated, however, that the toggle 40 can be made of other materials and can have other configurations so long as it can be seated inside the vessel against the vessel wall. The plug 88 can comprise a strip of compressible, resorbable, collagen foam and can be made of a fibrous collagen mix of insoluble and soluble collagen that is cross linked for strength. It should be appreciated, however, that the plug member 88 can have any configuration as desired and can be made from any material as desired. The suture 43 can be any elongate member, such as, for example a filament, thread, or braid.

Now referring to FIGS. 9A-9C, the guide wire 114 can be inserted through the puncture site 112 and into the vessel 13 such that a portion of the guide wire 114 protrudes from the vessel. Once the guide wire 114 is positioned, a proximal end of the guide wire 114 can be inserted into the distal end of the puncture locating dilator 110. As shown in FIG. 9A, the puncture locating dilator 110 can then be moved along the guide wire 114 until the distal end of the puncture locating dilator 110 and the distal port 42 enter the vessel 13 such that blood flows into the distal port 42 and out the proximal port 46 to thereby locate a position of the puncture site 112. The position of the puncture site 112 can be confirmed via feedback of blood flow exiting the blood proximal port 46 by alternatingly inserting and retracting the puncture locating dilator 110. As shown in FIG. 9A, after the position of the puncture site 112 has been located, the first visible marking 54a of the dilator 110 can be noted. That is, the first visible marking 54a that is adjacent the patient's skin can be noted. It should be appreciated, that in some embodiments, the puncture locating dilator 110 can be positioned over the guide wire 114 prior to the guide wire being inserted into the vessel 13.

Referring to FIG. 9B, the puncture locating dilator 110 may be utilized with an access sheath 23 that is also configured to be moved along the guide wire 114 toward the puncture site 112 and into the vessel 13 so as to further dilate the puncture site 112 and subsequently provide access to the vessel 13. The access sheath 23 can then receive a vascular closure device 12 that is configured to seal the puncture site 112. It should be appreciated, however, that the system can include additional dilators that have cross-sectional diameters that are different (e.g. greater) than the diameter of the puncture locating dilator 110 but less than that of the access sheath 23 so that the puncture site 112 can be gradually dilated and prepared for the access sheath 23. Both the puncture locating dilator 110 and the access sheath 23 may include respective depth markings that are configured to aid in locating the puncture site 112.

As shown in FIG. 9B, after the puncture locating dilator 110 has been removed from the guide wire 114 and any subsequent dilators have been removed, the access sheath 23 can be moved along the guide wire 114 toward the puncture site 112 such that the distal end of the access sheath 23 enters the vessel 13 through the puncture site 112. In particular, the proximal end of the guide wire 114 is inserted into the distal end of a sheath dilator 164. And then the sheath body 160 and sheath dilator 164 can be moved together along the guide wire 114 toward the puncture site 112. Once inserted, the sheath dilator 164 can be pulled proximally such that the sheath dilator 164 is removed from the access channel 168.

After the sheath dilator 164 has been removed, a vascular closure procedure can be performed through the access channel 168. Therefore, a closure device 12 can be moved into the access channel 168 until a distal portion 192 (e.g. at least a portion of the toggle 40) of the closure device 12 is distal to the distal end of the sheath body 160. As shown in FIG. 9C the access sheath 23 can then be moved such that a first visible marking 172a of the sheath body 160 that is visible adjacent the patient's skin corresponds with the noted first visible marking 54a of the puncture locating dilator 110. It should be appreciated, that the closure device 12 can be moved into the access channel 168 either prior to or after the positioning of the access sheath 23 such that the first visible marking 172a corresponds to the noted marking 54a. When the access sheath 23 is properly positioned, the closure device 12 will be positioned such that the sealing procedure can be completed. It should be appreciated, that while in the illustrated embodiment, the depth markings 172 are on the sheath body 160, in some embodiments, the depth markings can be on the closure device 12, as desired. Furthermore, it should be appreciated, that in such embodiments, the access sheath 23 can be pulled completely out of the vessel 13 when the closure device 12 is properly positioned.

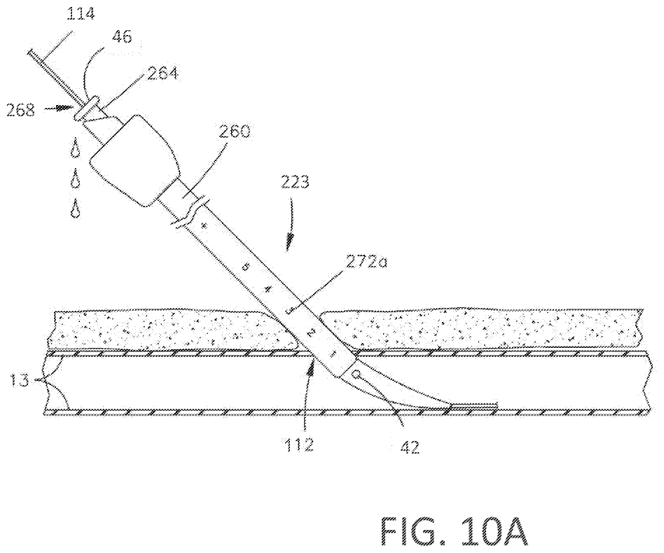

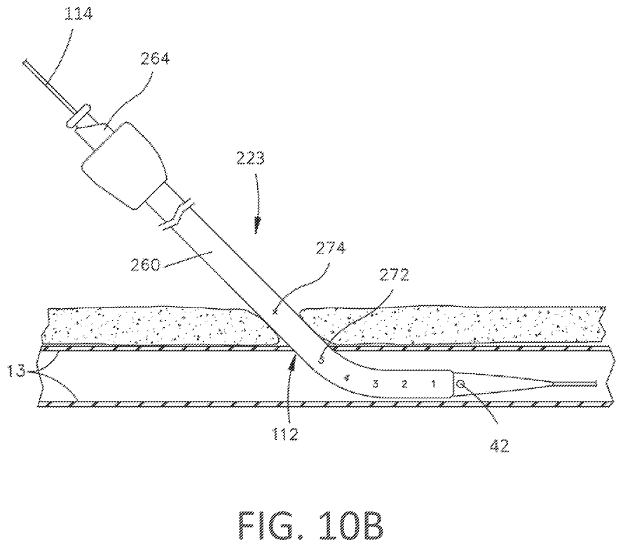

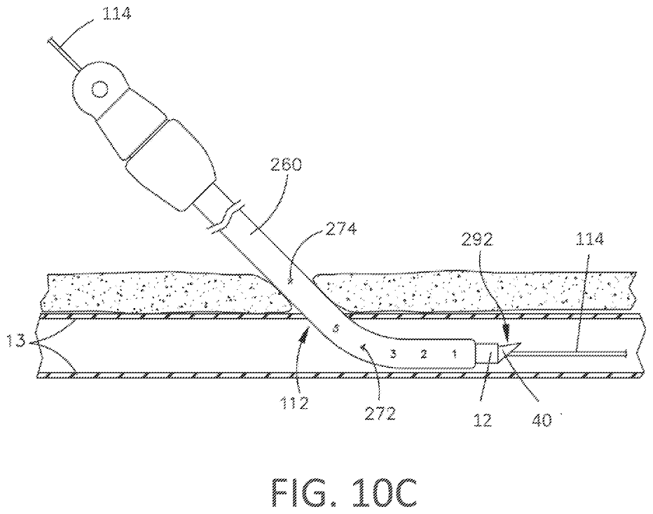

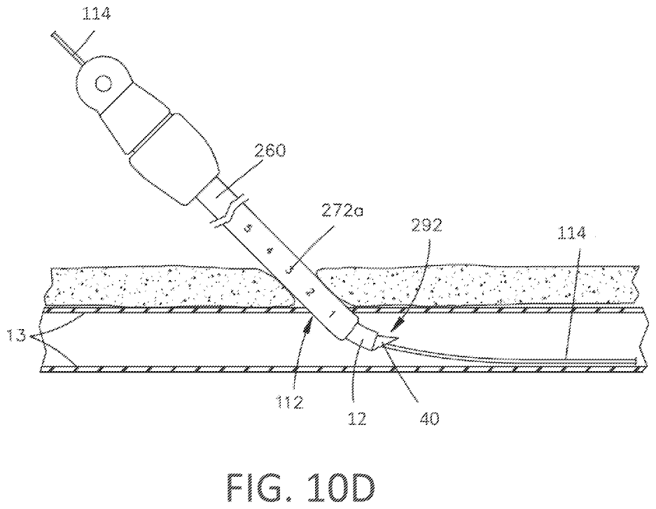

Now referring to FIGS. 10A-10D, in another embodiment, the guide wire 114 can be inserted through the puncture site 112 and into the vessel 13 such that a portion of the guide wire 114 protrudes from the vessel 13. Once the guide wire 114 is positioned, a proximal end of the guide wire 114 can be inserted into the distal end of a sheath dilator 264 of an access sheath 223. The access sheath 223 may have a similar structure and functionality as the access sheath 23. As shown in FIG. 8A, the sheath dilator 264 along with the sheath body 260 can then be moved along the guide wire 214 until the distal end of the sheath dilator 264 and the distal port 42 enter the vessel 13 such that blood flows into the distal port 42 and out the proximal port 46 to thereby locate a position of the puncture site 112. The position of the puncture site 112 can be confirmed via feedback of blood flow exiting the blood proximal port 46 by alternatingly inserting and retracting the sheath dilator 264 and sheath body 260 combination. As shown in FIG. 10A, after the position of the puncture site 112 has been located, a first visible marking 272a of the sheath body 260 can be noted. That is, a first visible marking 272a that is adjacent the patient's skin can be noted. It should be appreciated, that in some embodiments, the access sheath 223 can be positioned over the guide wire 114 prior to the guide wire 114 being inserted into the vessel 13.

As shown in FIG. 10B, after the first visible depth marking 272a has been noted, the access sheath 223 can be further moved along the guide wire 114 until a full insertion marker 274 is adjacent the patient's skin surface. At this time, the sheath dilator 264 can be pulled proximally and removed from the access channel 268. And after the sheath dilator 264 has been removed, a vascular closure procedure can be performed through the access channel 268. Therefore, as shown in FIG. 10C the closure device 12 can be moved into the access channel 268 until a distal portion 292 of the closure device 12 is distal to the distal end of the sheath body 260 and the closure device 12 couples to the sheath body via for example a snap fit. For example, at least a portion of a toggle 40 of the closure device 12 can be distal to the sheath body 260 when the closure device 12 is positioned within the access channel 268. As shown, in FIG. 10C, the closure device 12 can be moved along the guide wire 114 as it is being inserted into the access channel 268.

As shown in FIG. 10D, the sheath body 260 and closure device 12 can then be pulled proximally until the depth marking 272a noted during the puncture locating step becomes visible adjacent the patient's skin. When the access sheath 223, or at least the sheath body 260 is properly positioned, the closure device 12 will be positioned such that the sealing procedure can be completed. For example, the toggle 40 can be deployed into the vessel 13 so that the puncture site 112 can be sealed. It should be appreciated, that in some embodiments the closure device 12 can include the depth markings and the sheath body 260 can be pulled such that the sheath body 260 exits the vessel 13 and a first depth marking on the closure device 12 that corresponds to the noted depth marking is visible.

While the foregoing description and drawings represent the preferred embodiment of the present invention, it will be understood that various additions, modifications, combinations and/or substitutions may be made therein without departing from the spirit and scope of the present disclosure as defined in the accompanying claims. In particular, it will be clear to those skilled in the art that the present disclosure may be embodied in other specific forms, structures, arrangements, proportions, and with other elements, materials, and components, without departing from the spirit or essential characteristics thereof. One skilled in the art will appreciate that the present disclosure may be used with many modifications of structure, arrangement, proportions, materials, and components, which are particularly adapted to specific environments and operative requirements without departing from the principles of the present disclosure. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the present disclosure being indicated by the appended claims, and not limited to the foregoing description. It will be appreciated by those skilled in the art that various modifications and alterations of the present disclosure can be made without departing from the broad scope of the appended claims. Some of these have been discussed above and others will be apparent to those skilled in the art.

What is claimed:

1. A puncture location device configured to determine a location of a puncture in an artery relative to a skin surface of a patient, the puncture location device comprising:
a flexible elongated body that extends along a central longitudinal axis, the flexible elongated body having:
a proximal end,
a distal end spaced from the proximal end along the central longitudinal axis,
an inner channel that extends from the proximal end toward the distal end along the central longitudinal axis,
an inner core that defines the inner channel, the inner core including a first polymeric material, and
an outer layer that surrounds the inner core, the outer layer including a second polymeric material that is different from the first polymeric material;
the flexible elongated body further having at least one distal port that is open to the inner channel and that extends through the outer layer and the inner core, and a proximal port open to the inner channel and positioned between the proximal end and the at least one distal port,
wherein the at least one distal port is configured to receive blood therethrough such that blood travels through the inner channel and through the proximal port when the at least one distal port is placed in a path of blood flow.

2. The puncture location device of claim 1, wherein the first polymeric material comprises low-density polyethylene and the second polymeric material comprises high-density polyethylene.

3. The puncture location device of claim 1, wherein the first polymeric material is predominantly low-density polyethylene and the second polymeric material is predominantly high-density polyethylene.

4. The puncture location device of claim 1, wherein the flexible elongated body includes a plurality of markings spaced apart along a longitudinal direction that is parallel to the central longitudinal axis.

5. The puncture location device of claim 4, wherein the plurality of markings are etched into the outer surface of the outer layer.

6. The puncture location device of claim 4, wherein the plurality of markings are not etched into the inner core.

7. The puncture location device of claim 1, wherein the inner core has a greater flexibility than the outer layer.

8. The puncture location device of claim 1, wherein the flexible elongated body has an outer cross-sectional dimension that is perpendicular to the central longitudinal axis, wherein the outer cross-sectional dimension is at least 0.18 inches.

9. The puncture location device of claim 1, wherein the outer layer comprises between 0.085 and 0.0115 inches of a radius of the flexible elongated body.

10. The puncture location device of claim 9, wherein the outer layer comprises about 0.01 inches of the radius of the flexible elongated body.

11. The puncture location device of claim 1, wherein the inner core comprises between 0.0440 and 0.060 inches of a radius of the flexible elongated body.

12. The puncture location device of claim 11, wherein the inner core is about 0.052 of the radius of the flexible elongated body.

13. The puncture location device of claim 1, wherein the inner core and the outer layer comprise between 65% and 80% of an outer diameter of the flexible elongated body.

14. The puncture location device of claim 1, wherein the inner core and the outer layer comprise about 75% of an outer diameter of the flexible elongated body.

15. The puncture location device of claim 1, wherein the outer layer comprises up to about 10% of an outer diameter of the flexible elongated body.

16. The puncture location device of claim 1, wherein the outer layer comprises between about 3% and 8% of an outer diameter of the flexible elongated body.

17. The puncture location device of claim 1, wherein the inner core comprises between about 20% and 35% of an outer diameter of the flexible elongated body.

18. The puncture location device of claim 1, wherein the inner core comprises between about 25% and 30% of an outer diameter of the flexible elongated body.

19. The puncture location device of claim 1, wherein the flexible elongated body has a tapered portion that defines the distal end and a linear portion that extends from the tapered portion to the proximal end, wherein the tapered portion tapers toward the central longitudinal axis.
